(19) United States
(12) Patent Application Publication
(10) Pub. No.: US 2024/0423720 A1
(43) Pub. Date: Dec. 26, 2024

(54) Method And System Of Visualization During Orthopaedic Surgery

(71) Applicant: Akunah Medical Technology Pty Ltd, Greenslopes, Brisbane (AU)

(72) Inventors: Ashish Gupta, Brisbane (AU); Marine Mathilde Madeleine Launay, Brisbane (AU)

(21) Appl. No.: 18/705,255

(22) PCT Filed: Oct. 28, 2021
(86) PCT No.: PCT/AU2021/051256

§ 371 Date: Apr. 26, 2024

(51) Int. Cl. A61B 34/10; A61B 17/56; A61B 34/00; A61B 90/00; G06T 7/00; G06T 19/00

(52) U.S. Cl. CPC A61B 34/10; A61B 17/56; A61B 34/25; A61B 90/37; G06T 7/0012; G06T 19/006; A61B 2034/102; A61B 2034/105; A61B 2034/107; A61B 2090/365; A61B 2090/366; G06T 2207/20172; G06T 2207/30008; G06T 2210/41

(57) ABSTRACT

A method of visualization during orthopaedic surgery. The method includes the step of a pre-operative planning step comprising displaying an initial three-dimensional image depicting anatomical features of a patient's bone. The image is retrieved by undertaking imaging or scanning of the patient's bone. The image is processed using a processor operable to carry out one or more image processing steps to generate a modified three-dimensional image. A mixed reality visualization device receives the modified three-dimensional image. The method also includes the step of displaying the modified three-dimensional image, via the mixed reality visualization device, and overlaying the modified three-dimensional image on or adjacent the anatomical features of the patient's bone to provide a visual guide to a surgeon while conducting surgery.

### TECHNICAL FIELD

The present invention relates to a method and system of visualization during orthopaedic surgery. In particular, the present invention relates to a method and system of visualization of a shoulder joint during surgery, although the invention is not to be taken as limited solely to this application.

### BACKGROUND

Any references to methods, apparatus or documents of the prior art are not to be taken as constituting any evidence or admission that they formed, or form part of the common general knowledge.

The shoulder is one of the most complex joints in the human body being an intricate combination of four different joints participating simultaneously in the motion of the shoulder girdle.

Shoulder surgery repairs a damaged, degenerated or diseased shoulder joint. It is a treatment for a variety of diseases and conditions in the shoulder joint. These commonly include rotator cuff tears, shoulder dislocations, and shoulder separations. Shoulder surgery can potentially help restore pain-free range of motion and full function to a damaged shoulder joint. However, due to the complexity of the joint, surgery can often be difficult.

Accurate implant positioning is crucial to ensure a successful outcome of shoulder arthroplasty. Malpositioning of the glenoid component can lead to scapular notching, implant loosening or instability. Significant glenoid bone defects can be encountered in patients with severe osteoarthritis, previous failed shoulder replacement procedure, cuff tear arthropathy with glenoid erosion, or chronic glenohumeral dislocation. Intraoperative view of the scapula is limited, and difficult surgical exposure pose significant challenges for the orthopaedic surgeon intraoperatively. Preoperative evaluation of scapular and glenoid anatomy and surgical planning are crucial steps to ensure successful postoperative outcomes.

Understanding of shoulder pathology has increased significantly in the last decade and technological developments have allowed for enhanced preoperative planning solutions & innovative tools for surgical execution.

In particular, technology has allowed orthopaedic surgeons to shift from plain radiographs & CT scans to 3D planning using specialised software. However, execution of the preoperative plan to reproduce the exact intraoperative implant position remains elusive. The use of intraoperative computer navigation such as CAOS (computer-assisted orthopaedic surgery) & PSI (patient-specific instrumentation) were developed to help bridge this gap.

Use of intraoperative computer navigation assists in implant positioning. However, the operating time can be significantly longer using CAOS. 3D printed PSI is an alternative to assist in implant positioning intraoperatively, from a preoperative plan created in silico. Customised guidewire positioning allows the surgeons to reproduce the glenoid baseplate position as planned preoperatively. Both CAOS & PSI involve additional steps in medical journey of care & increase associated costs.

More recently, Mixed Reality (MR) technology has been employed for preoperative planning & intraoperative adjuncts. MR allows the surgeon to view virtual models throughout the surgery using a head-mounted display (HMD), which aids in understanding the anatomy & implant positioning.

There is also a gap to link the preoperative planning with recommendations based on clinical outcomes. In cases of significant & complex glenoid bone defects, such as revision surgeries, a 2-stage procedure may be recommended. The first stage allows for primary implant removal, assessment & perhaps grafting of the glenoid bone defect or implantation of a cement spacer before the second stage. This process is highly invasive and may induce significant morbidity to the patient and increases the risks of complications during re-surgery.

Attempts to provide a single stage surgical execution of a revision reverse shoulder arthroplasty (RSA) may require 3D printing of the scapular 3D model & several attempts in the lab to adjust for the prosthetic device & screws to achieve correct & reliable implant positioning, which can yield undesirable results.

### SUMMARY OF INVENTION

In an aspect, the invention provides a method of visualization during orthopaedic surgery, the method comprising:
a pre-operative planning step comprising displaying an initial three-dimensional image depicting anatomical features of a patient's bone, the initial three-dimensional image being retrieved by undertaking imaging or scanning of the patient's bone; and processing the initial three-dimensional image using a processor operable to carry out one or more image processing steps to generate a modified three-dimensional image;
providing a mixed reality visualization device which receives the modified three-dimensional image; and
displaying the modified three-dimensional image, via the mixed reality visualization device, and overlaying the modified three-dimensional image on or adjacent the anatomical features of the patient's bone to provide a visual guide to a surgeon while conducting surgery.

Preferably, the displaying step further comprises displaying the initial three-dimensional image via the mixed reality visualization device and overlaying the displayed initial image on or adjacent the anatomical features of the patient's bone to provide a visual guide to a surgeon while conducting surgery.

Preferably, the pre-operative planning step further comprises analysis of metal implants in an initial two-dimensional image of the bone using the processor and suppressing visualization of metal artefacts and generating the modified three-dimensional image of the bone. Preferably, the initial two-dimensional image is obtained from the imaging or scanning of the patient's bone. Preferably, the initial three-dimensional image is generated from the initial two-dimensional image.

Preferably, the analysis of metal implants in the initial two-dimensional image comprises applying a mask to the initial two-dimensional image and reducing scatter of the initial two-dimensional image.

Preferably, reducing scatter of the initial two-dimensional image comprises applying a filter and/or modifying a filter strength of the filter applied to the initial two-dimensional image.

Preferably, the pre-operative planning step further comprises analysis of a defect visible in the initial three-dimensional image using the processor and generating geometric dimensions for a bone graft to fill said defects and generating a three dimensional image of the graft; and wherein the displaying step comprises displaying the three-dimensional image of the graft via the mixed reality visualization device and overlaying the displayed three-dimensional image of the graft on or adjacent the anatomical features of the patient's bone to provide a visual guide to the surgeon while conducting surgery.

Preferably, the three-dimensional image of the graft is blended or merged with the initial three-dimensional image or the modified three-dimensional image.

Preferably, the pre-operative planning step further comprises generating a guidewire trajectory, and displaying the guidewire trajectory, via the mixed reality visualization device, overlayed on the displayed modified three-dimensional image to provide a visual guide to a surgeon while conducting surgery.

Preferably, the processor is operable to carry out one or more image processing steps.

Preferably, the processor generates one or more modified three-dimensional images from the initial three-dimensional image.

Preferably, the processor generates the initial three-dimensional image from one or more initial two-dimensional images of the patient's bone.

Preferably, the processor is operable to receive user input via a user input interface to carry out the one or more image processing steps to generate the modified three-dimensional image.

In another aspect, the invention provides a system comprising:
a processor operable to carry out image processing; and
a mixed reality visualization device which receives the modified three-dimensional image;
wherein the processor:
   receives an initial three-dimensional image depicting anatomical features of a patient's bone, the initial three-dimensional image being retrieved by undertaking imaging or scanning of the patient's bone;
   processes the initial three-dimensional image with one or more image processing steps to generate a modified three-dimensional image; and
   communicates the modified three-dimensional image to the mixed reality visualization device; and
wherein the mixed reality visualization device displays the modified three-dimensional image and overlays the modified three-dimensional image on or adjacent the anatomical features of the patient's bone to provide a visual guide to a surgeon while conducting surgery.

Preferably, the mixed reality visualization device displays the initial three-dimensional image and overlays the displayed initial three-dimensional image on or adjacent the anatomical features of the patient's bone to provide a visual guide to a surgeon while conducting surgery.

Preferably, the processor analyses a metal implant in the initial two-dimensional image and suppresses visualization of metal artefacts and generates the modified three-dimensional image of the bone.

Preferably, analysing the metal implant in the initial two-dimensional image further comprises the processor applying a mask to the initial two-dimensional image and reducing scatter of the initial two-dimensional image.

Preferably, reducing scatter of the initial two-dimensional image further comprises the processor applying a filter and/or modifying a filter strength of the filter applied to the initial two-dimensional image.

Preferably, the processor:
analyses a defect visible in the initial three-dimensional image;
generates geometric dimensions for a bone graft to fill said defects; and
generates a three dimensional image of the graft; and
wherein the mixed reality visualization device displays the three-dimensional image of the graft and overlays the displayed three-dimensional image of the graft on or adjacent the anatomical features of the patient's bone to provide a visual guide to the surgeon while conducting surgery.

Preferably, the processor blends or merges the three-dimensional image of the graft with the initial three-dimensional image or the modified three-dimensional image.

Preferably, the processor generates a guidewire trajectory and the mixed reality visualization device displays the guidewire trajectory overlayed on the displayed modified three-dimensional image to provide a visual guide to a surgeon while conducting surgery.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 illustrates a system for visualization of orthopaedic surgery according to an embodiment of the present invention;

FIG. 2 further illustrates the system of FIG. 1;

FIG. 3 illustrates a process diagram for identifying bone defects and generating a 3D image for display through a mixed reality device during surgery;

FIG. 4 illustrates another process diagram for suppressing metal artefacts in a patient image and generating a 3D image for display through a mixed reality device during surgery;

FIG. 5 illustrates generation of an overlay image from a pathological image and a contralateral image;

FIG. 6 illustrates a simplified registration technique for overlaying the pathological image and the contralateral image from FIG. 5;

FIG. 7 illustrates a graft creation process;

FIG. 8 further illustrates the graft creation process and the graft;

FIG. 9 illustrates an intraoperative visualization of 3D pre-planned holograms for patient-specific humeral allograft;

FIG. 10 shows an intraoperative visualization of pre-planned 3D holograms and a matched surgical execution;

FIG. 11 illustrates a mask applied to an image of bone having metal implants to identify and isolate the metal implant from the surrounding bone;

FIG. 12 illustrates a preoperative axial CT scan with significant metal artefacts;

FIG. 13 illustrates use of a scatter reduction tool with 50% filtering strength (filtered image-right; unfiltered image-left);

FIG. 14 illustrates a 3D model of a glenoid with guidewire trajectory; and

FIG. 15 illustrates an intraoperative view of a glenoid bone defect with guidewire matching 3D hologram.

### DETAILED DESCRIPTION OF PREFERRED EMBODIMENTS

The present disclosure, generally, relates to a method of visualization during orthopaedic surgery. The method, in a first step, includes a pre-operative planning step where an initial three-dimensional image depicting anatomical features of a patient's bone is displayed. The initial three-dimensional image is, generally, retrieved or obtained by undertaking imaging or scanning of the patient's bone.

The initial three-dimensional image then undergoes image processing using a processor operable to carry out image processing techniques to then generate one or more modified three-dimensional images. In some embodiments, user input is provided through a user input interface to assist with the generation of the modified three-dimensional images.

In a second step, the method provides a mixed reality visualization device which receives the modified three-dimensional image. In one embodiment, the mixed reality visualization device is in communication with the processor. The mixed reality visualization device receives and displays the modified three-dimensional image and overlays the displayed one or more modified images on or adjacent the anatomical features of the patient's bone to provide a visual guide to a surgeon while conducting surgery.

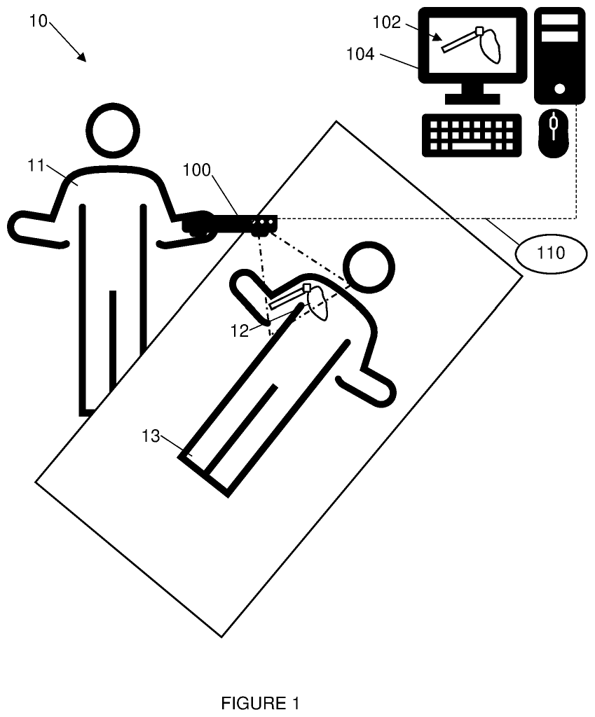

Referring now to FIGS. 1 to 4, a system 10 for visualization during orthopaedic surgery which provides a guide for a surgeon is shown.

In FIG. 1, a surgeon 11 (or a clinician, or other suitably qualified person), scans a bone 12 of a patient 13 using a medical imaging device 100, capable of medically imaging the anatomy of the patient 13. While the medical imaging device 100 is shown as a handheld device in the illustration, it is expected that the medical imaging device 100 will take the form of a Computed Tomography (CT) scanner or similar apparatus.

The medical imaging device 100 generates initial two-dimensional images 110 of the bone 12. An initial three-dimensional image 102, depicting anatomical features of the bone 12, is generated from the scan and the initial two-dimensional images 110.

The initial three-dimensional image 102 is represented digitally on an medical image processing computer 104 having a processor 105 which facilitates the pre-operative planning step described above.

In particular, the medical image processing computer 104 displays the initial three-dimensional image 102 depicting the anatomical features of the bone 12 of a patient 13 and is configured to process the initial three-dimensional image 102 by executing image processing steps in conjunction with optional user input provided by the surgeon 11 (or other clinician, medical specialist or team of specialists) through a user input interface of the medical image processing computer 104. The image processing steps performed by medical image processing computer 104 will be described in more detail below.

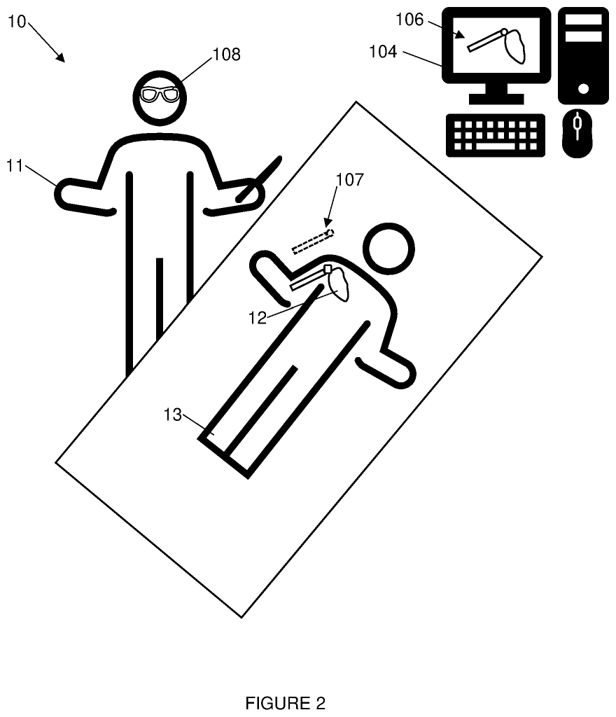

As a result of the above, a modified three-dimensional image 106, shown in FIG. 2, is produced.

The modified three-dimensional image 106 (or part thereof) is then provided as a virtual image 107 (in the form of a hologram, for example) for viewing by surgeon 11 during surgery conducted on patient 13 through a mixed reality visualization device 108 (such as a Microsoft HoloLens 2, for example) which is in communication with the medical image processing computer 104.

The mixed reality visualization device 108 displays the modified three-dimensional image 106 as the virtual image 107 and overlays the virtual image 107 (or part of the modified three-dimensional image 106 or a series of images) on or adjacent the anatomical features of the bone 12 of patient 13 to provide a visual guide to the surgeon 11 while conducting surgery, as shown in FIG. 2.

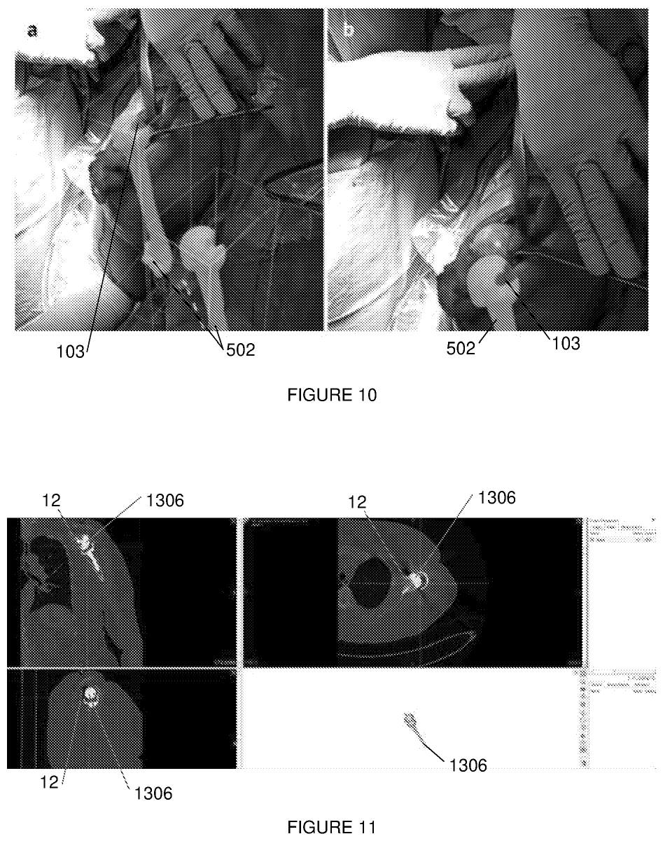

In some embodiments, a virtual image of both the initial three-dimensional image 102 and the modified three-dimensional image 106 may be displayed to the surgeon 11 (see FIG. 10, for example).

### Bone Defect Analysis Example

Returning to the medical image processing computer 104, the medical image processing computer 104 is configured to analyse defects in the initial three-dimensional image 102 of the bone 12 of the patient 13 and generate geometric dimensions for one or more bone grafts to fill said defects. This is particularly useful for identifying defects, and analysing and developing a graft for bone defects in the humeral head.

From this analysis and generated geometric dimensions of the bone grafts, the medical image processing computer 104 generates a three-dimensional image of a graft 103 to repair the bone 12. Subsequently, in the visualization process described above, the medical image processing computer 104 communicates the three-dimensional image of the graft to the mixed reality visualization device 108 and overlays the three-dimensional image of the graft 103 on or adjacent the bone of the patient to provide a visual guide to the surgeon while conducting surgery. In some embodiments, the three-dimensional image of the graft 103 may be combined with the initial three-dimensional image 102 of the bone 12 to thereby generate the modified three-dimensional image 106 of the bone 12 that is presented to the surgeon.

An example of the image processing steps undertaken to identify and analyse the bone defect, and generate a geometrically suitable graft will now be described.

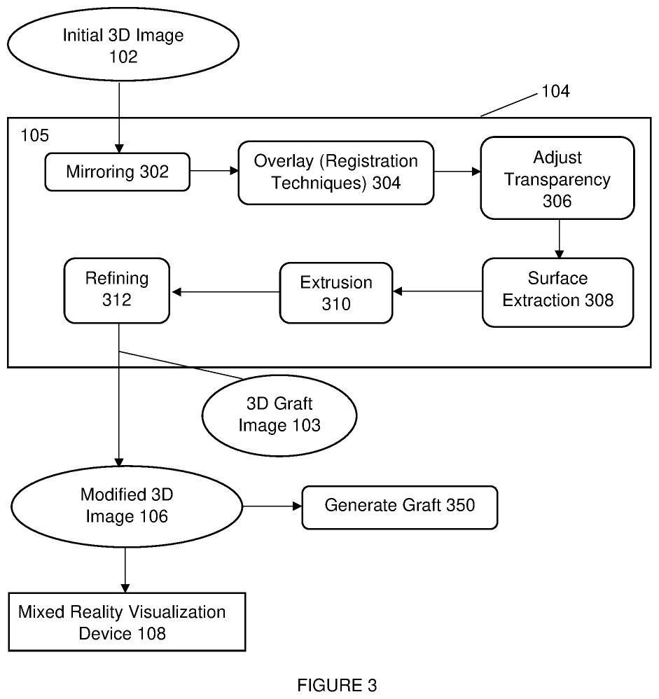

The following example references FIG. 3 which illustrates the process as a series of steps carried out by the processor 105 of the medical image processing computer 104.

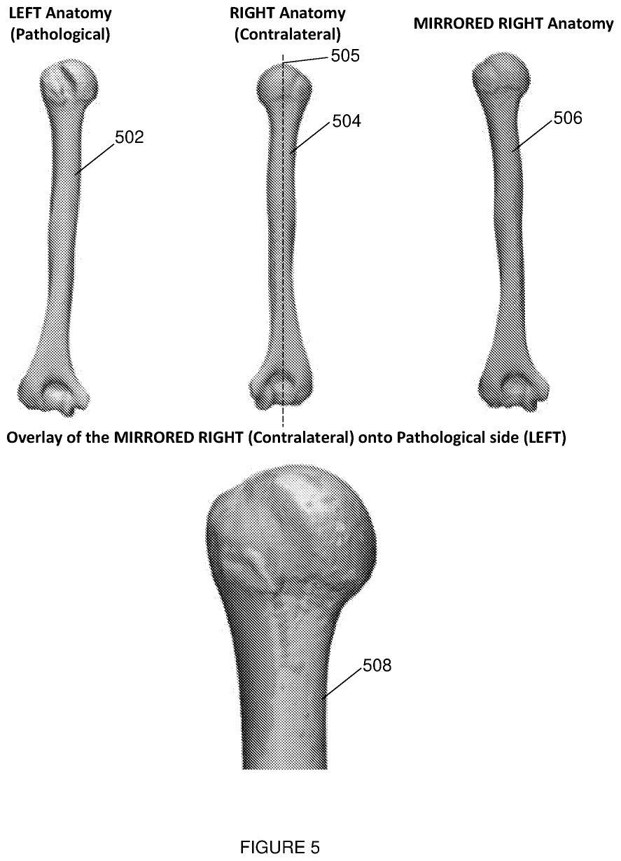

With reference to FIG. 5, initial three-dimensional images 102 of the left anatomy (the pathological side) and the right anatomy (the contralateral side) are obtained to provide a left anatomy image 502 and a right anatomy image 504. Use of the contralateral healthy side will allow for comparison and estimation of the native, pre-morbid anatomy of the pathological side.

The left anatomy image 502 shows a defect 503 in the form of a Hill Sachs lesion.

It is assumed that sufficient imaging of the contralateral side is available and that it can reasonably be considered as healthy. Otherwise, other methods may need to be utilised.

Assuming the image of the contralateral side (i.e. right anatomy image 504) is suitable, a mirroring step 302 is performed by the medical image processing computer 104 where the right anatomy image 504 is mirrored about a central vertical axis 505 to create a mirrored right anatomy image 506. In some embodiments, the anatomy can be mirrored about a pre-defined plane (preferably a vertical plane), rather than an axis, to create an opposing anatomy image.

As noted above, this mirrored right anatomy image 506 forms the basis for comparison and estimation of how the pathological side would appear, if it were healthy.

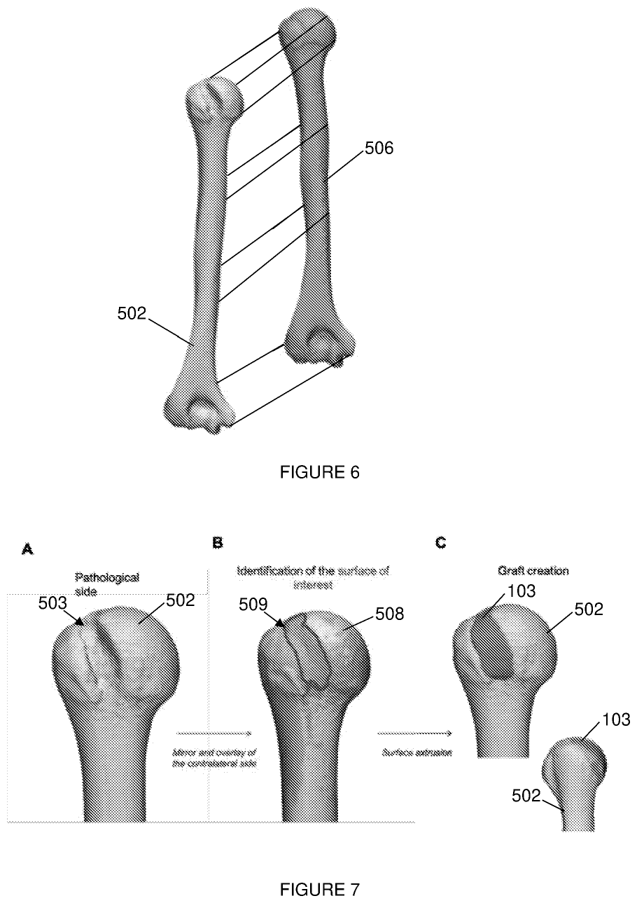

The medical image processing computer 104 then performs an overlay step 304. The mirrored right anatomy image 506 and the left anatomy image 502 are overlayed or superimposed to generate an overlay image 508. This overlay step 304 is performed using registration techniques, whereby registering two parts is performed by selecting N points on the fixed part (i.e. the pathological side/left anatomy image 502) that will need to correspond to the same number of points of the other part (i.e. the mirrored right anatomy image 506). An example of the registration is shown in FIG. 6 using the left anatomy image 502 and mirrored right anatomy image 506.

The overlayed image 508 is then analysed to identify a region of interest 509 (areas of the bone with the defect 503) and perform surface extraction.

First, identification of the defect from the overlayed image 508 is performed. Identification is performed by increasing the transparency of the mirrored right anatomy image 506 to enable the region of interest 509 requiring graft creation to be identified. This is performed at step 306.

Next, a surface extraction step 308 is performed. The bony surface of the mirrored right anatomy image 506 which corresponds to the region of interest 509 identified above is marked and extracted. This is shown in step B of FIG. 7.

Generally, marking is performed by software which selects triangles of interest from the three-dimensional model and isolating those triangles of interest onto a separate surface containing only the triangles.

Following the identification of the region of interest 509 and extraction of said regions of interest, a graft to fill the defect can be planned and generated.

Creating the graft from the regions of interest includes three steps: (1) an extrusion of the region of interest step 310; (2) a refining step 312; and (3) additional considerations. Step (1) is essential, but steps (2) and (3) may be omitted in some embodiments.

Extrusion of the region of interest 509 is performed by taking the image of the region of interest 509 and extruding the image until the extrusion meets the surfaces of the cavity of the defect 503 in the left anatomy image 502 (i.e. the pathological side). This process creates a 3D volume in the form of a three-dimensional image of a graft 103 that fills the bone defect 503 identified in the identification step described above. The extrusion is typically performed by a software program where the direction of the extrusion and refinement can be manually performed or assisted by a user.

In image C of FIG. 7 the 3D volume defining the three-dimensional image of the graft 103 can be seen from multiple angles.

Moving to step (2), further refining can be achieved by importing the contours of the three-dimensional image of the graft 103 onto the patient's medical images (i.e. the image of the pathological side-left anatomy image 502).

Manual editing of the contours of the graft may be performed as necessary to match as close as possible the specific bony defect of the patient.

In step (3), an export of the refined (or unrefined) three-dimensional image of the graft 103 corresponds to the preoperatively planned bone graft that will be used during surgery. That is, the three-dimensional image of the graft 103 is used to create the preoperatively planned bone graft 350. Geometric measurements (e.g. length, width, depth, diameter, radius of curvature, etc.) can also be extracted from the three-dimensional image of the graft 103.

The above is one example of how a graft can be generated. It will be understood that other, well-known graft generating methods can be used.

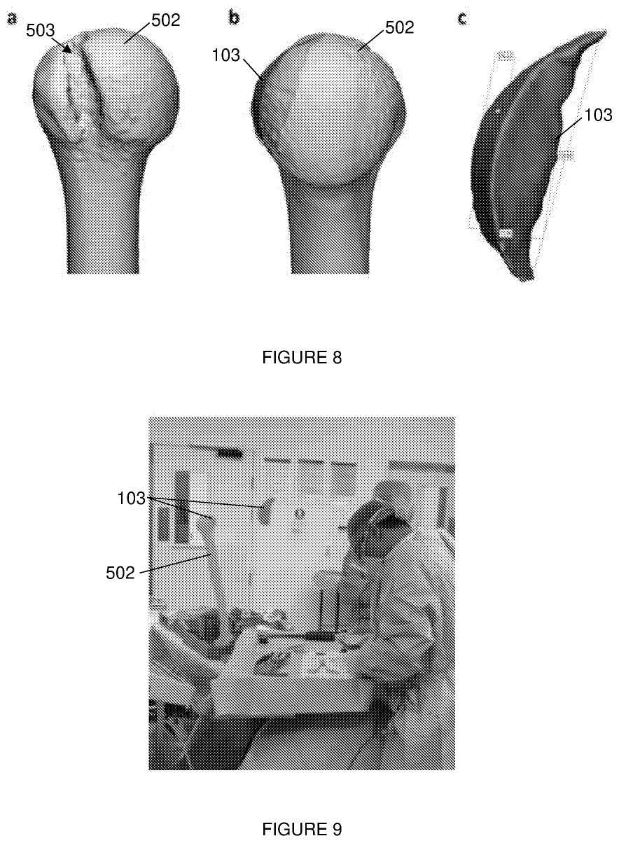

As an example, a 19-year-old patient presents with a first-time traumatic left shoulder dislocation. Analysis of CT scans reveals a large Hill Sachs lesion requiring surgery to prevent recurrent shoulder instability. Using the systems and methodology described above, 3D models of the patient's anatomy and a preoperative plan of the size and shape of a customised patient-specific graft (See FIG. 8, which is similar to FIG. 7 but shows the 3D volume defining the graft in isolation from the bone) were achieved. A size-matched humeral allograft was used. Utilising a mixed reality visualization device, a surgeon is able to visualise the pre-planned 3D holograms in real-time during surgery. This allows for real-time visualisation of the humeral defect, and graft shape and dimensions (See FIGS. 9 and 10). Use of preoperative patient-specific planning enables the crafting of a personalised graft whilst the hologram is overlayed on the donor allograft. Real-time visualisation of 3D holograms in the operating room allows the surgeon to match the pre-planned shape of the allograft without requiring 3D printing.

### Reverse Shoulder Arthroplasty Example—Glenoid Analysis

In another embodiment, the medical image processing computer 104 is configured to analyse the bone of the patient which has metal implants (such as in the case of reverse shoulder arthroplasty). The medical image processing computer 104 identifies and suppresses visualisation of the metal artefacts caused by metal implants in an initial two-dimensional image 110 and then generates the initial three-dimensional image 102 and a modified three-dimensional image 106 of the bone 12. This is particularly useful for analysing the glenohumeral joint that has been the subject of previous shoulder arthroplasty and identifying and grafting bone defects in the glenoid.

An example of the image processing steps undertaken to analyse the glenohumeral joint to remove or suppress the presence of metal artefacts in the images will be described. The Inventors utilised Mimics software (Materialise) in the following example.

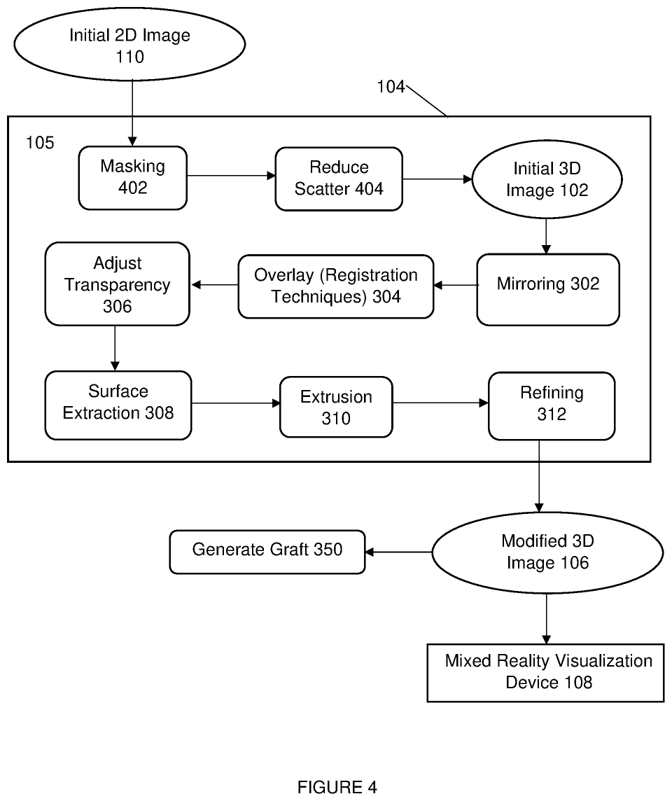

The following example references FIG. 4 which illustrates the process as a series of steps carried out by the processor 105 of the medical image processing computer 104.

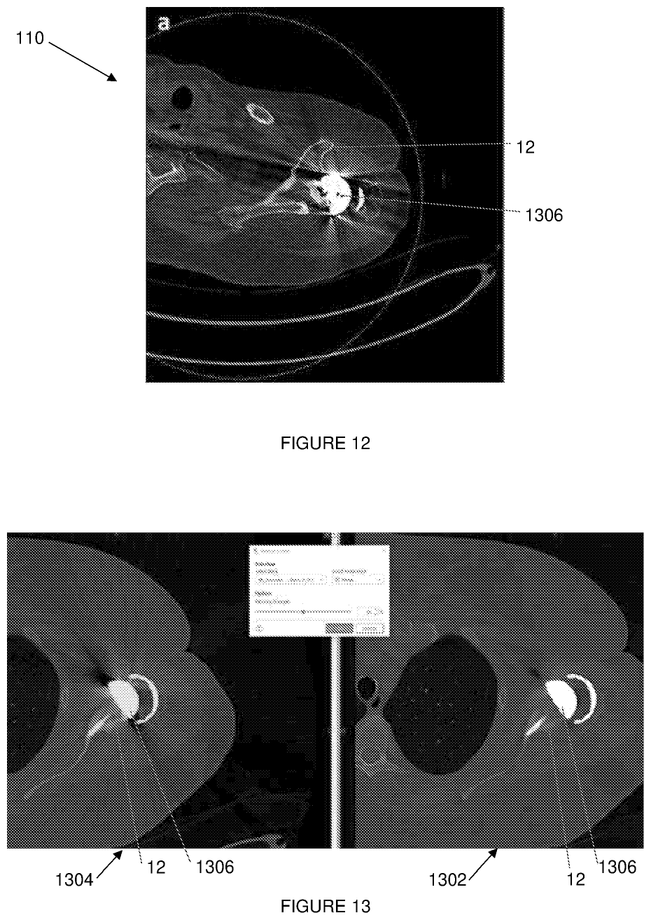

Revisions of RSA (reverse shoulder arthroplasty) are particularly challenging surgical procedures. Removal of the primary implant is often associated with significant glenoid bone loss. Complex bone defects and joint line medialisation necessitate customised grafts or metallic augments. However, presence of the primary metallic prosthesis in the preoperative CT scans creates significant artefact, which prevents evaluation of the size and extent of the glenoid bone defect preoperatively (see FIG. 12). At present, the surgeon must assess the glenoid anatomy and extent of the defect during surgery. In cases of significant and complex glenoid bone defects, a 2-stage procedure is recommended. The first stage allows for primary implant removal, assessment and perhaps grafting of the glenoid bone defect and implantation of a cement spacer (if required) before the second stage. Postoperative CT images (with the implants now removed) help guide the second stage of the arthroplasty, which consists of implanting new prosthetic devices.

As described above, 2-stage procedures, although the standard of care, do induce significant morbidity to the patient and increase the risks of complications during re-surgery. Personalised metal artefact reduction CT segmentation (as described in the present disclosure) enables the surgeon to achieve a single stage revision RSA by combining preoperative planning and intraoperative visualisations using mixed reality.

The identification and suppression of metal artefacts is achieved in a two step process including a masking step 402 which applies a mask to the initial two-dimensional image 110 received from medical imaging device 100 and a reduce scatter step 404 which reduces scatter in the initial two-dimensional image 110 using filtering techniques.

First, in the masking step 402 executed by medical image processing computer 104, a mask is applied to the initial two-dimensional image 110. In manual segmentation, a mask represents the voxels (3D pixels) of interest that are to be segmented into a specific region of interest or separated from other regions of interest.

Masking is a well-known image processing technique which effectively identifies a unique shape or characteristic of an object of interest in an image that is to be isolated or emphasised from the rest of an image and then isolates or emphasises that object of interest. As such, the masking step allows the metal implants 1306 to be identified and isolated in the initial two-dimensional image 110 of the bone 12 (see FIGS. 11 and 13). In effect, the mask highlights the metal implant 1306 so that it can be visually separated from the surrounding bone 12.

After the mask has been applied, the reduce scatter step 404 is performed. Scatter radiation present in the image is reduced by applying a filter and adjusting filtering strength. Scatter occurs when radiation impacts and deflects off an object (such as a metal implant, for example). Scatter is detrimental to image quality as it adds unwanted exposure to the image and decreases radiographic contrast without contributing any valuable patient information. Thus, it is important that scatter be reduced as much as possible.

The medical image processing computer 104 applies a filter strength of 50% as a starting point. The image with the filter applied can be visually assessed to assess the balance between efficient metal artefact reduction and detectability of the bony anatomy boundaries. Depending on the outcome of the initial filtering, the filter strength may need to be adjusted in increments of approximately 5%-10% until a satisfactory balance is achieved. An example of a satisfactorily balanced 2D image 1302 (right) compared to non-filtered 2D image 1304 (left) is shown in FIG. 13, where metal implant 1306 has been identified and masked and scatter has been reduced in the image. As can be seen, the scatter emanating from about the metal implant 1306 is significantly reduced in satisfactorily balanced 2D image 1302 as compared to non-filtered 2D image 1304. In particular, the bone boundaries remain visible enough to allow for manual and/or automatic segmentation.

As illustrated in FIG. 4, in some embodiments, steps 402 and 404 can be performed in advance of the graft forming steps 302-312 described above.

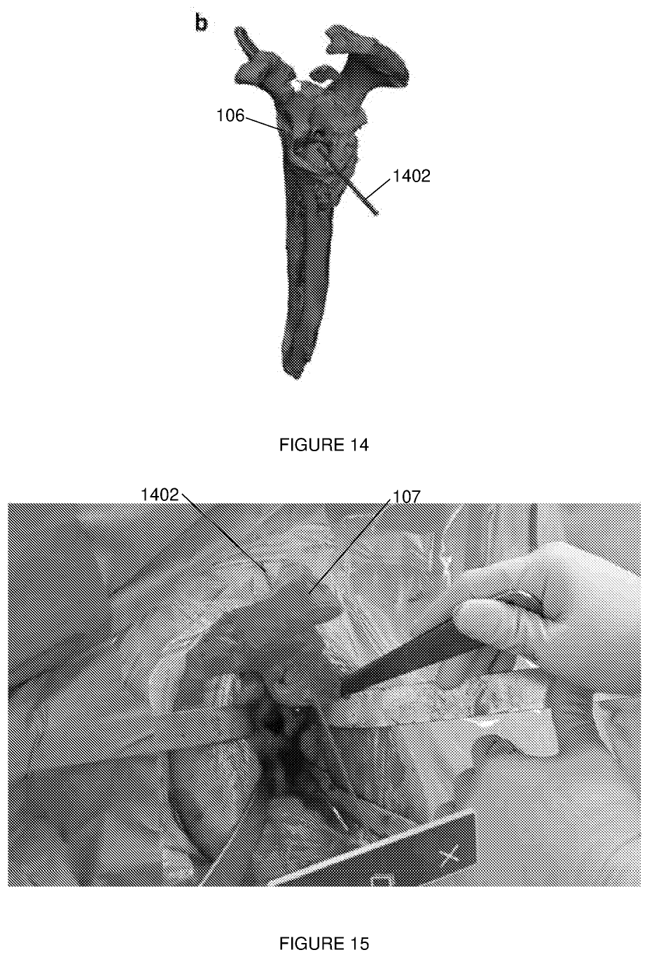

In some embodiments, the medical image processing computer 104 generates a guidewire trajectory 1402 that is displayed to the surgeon via the mixed reality visualization device 108, by overlaying the guidewire trajectory 1402 on the virtual image 107 based on the modified three-dimensional image 106 of the bone to provide a visual guide to a surgeon while conducting surgery, as shown in FIGS. 14 and 15.

Utilising the techniques described herein, a preoperative planning phase facilitates the creation of 3D models of a shoulder joint and to subtract metal artefacts and primary implants to allow the surgeon to ascertain the amount and extent of glenoid bone loss and any bone defects preoperatively. 3D modelling of the complete scapula and collaboration between the surgeon and the engineering team enables pre-planning of the guidewire trajectory to maximise reliance on remaining viable bone stock (see FIG. 14). Use of a mixed reality headset allows the surgeon to visualise a 3D hologram of the glenoid and corresponding guidewire intraoperatively, which assists in baseplate and screws positioning (see FIG. 15). Embodiments of the present invention which provide a single stage surgical execution mitigate the need for 3D printing of the scapular 3D model and attempts in the lab to achieve correct and reliable implant positioning.

Embodiments of the invention provide a digitised toolkit for surgeons allowing CT scan segmentation & metal artefact reduction, enhanced glenoid defect assessment & virtual surgical protocols, which can be used to guide the surgeon through the use of MR. This aims to provide a bridge between preoperative planning, surgical execution, and clinical outcomes.

Embodiments of the invention also provide a platform which integrates analysis of the shoulder, preoperative planning, surgical simulation & virtual intraoperative patient specific guidance employing MR. The Inventors envision that this has the potential to allow preoperative planning where in revision shoulder arthroplasty only one procedure is required to remove the original prosthesis, undertake essential reconstruction & insert a new prosthetic device.

In this specification, adjectives such as first and second, left and right, top and bottom, and the like may be used solely to distinguish one element or action from another element or action without necessarily requiring or implying any actual such relationship or order. Where the context permits, reference to an integer or a component or step (or the like) is not to be interpreted as being limited to only one of that integer, component, or step, but rather could be one or more of that integer, component, or step, etc.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

1. A method of visualization during orthopaedic surgery, the method comprising:
a pre-operative planning step comprising displaying an initial three-dimensional image depicting anatomical features of a patient's bone, the image being retrieved by undertaking imaging or scanning of the patient's bone; and
processing the image using a processor operable to carry out one or more image processing steps to generate a modified three-dimensional image;
providing a mixed reality visualization device which receives the modified three-dimensional image; and
displaying the modified three-dimensional image, via the mixed reality visualization device, and overlaying the modified three-dimensional image on or adjacent the anatomical features of the patient's bone to provide a visual guide to a surgeon while conducting surgery.

2. A method in accordance with claim 1 wherein the displaying step further comprises displaying the initial three-dimensional image via the mixed reality visualization device and overlaying the displayed initial three-dimensional image on or adjacent the anatomical features of the patient's bone to provide a visual guide to a surgeon while conducting surgery.

3. A method in accordance with claim 1 wherein the pre-operative planning step further comprises analysis of metal artefacts in the initial three-dimensional image using the processor and suppressing visualization of the metal artefacts and generating the modified three-dimensional image of the bone.

4. A method in accordance with claim 3 wherein the analysis of metal artefacts in the initial three-dimensional image comprises applying a mask to an initial two-dimensional image and reducing scatter of the initial two-dimensional image.

5. A method in accordance with claim 4 wherein reducing scatter of the initial two-dimensional image further comprises the processor applying a filter and/or modifying a filter strength of the filter applied to the initial two-dimensional image.

6. A method in accordance with claim 1 wherein the pre-operative planning step further comprises analysis of a defect visible in the initial three-dimensional image using the processor and generating geometric dimensions for a bone graft to fill said defects and generating a three dimensional image of the graft; and
wherein the displaying step comprises displaying the three-dimensional image of the graft via the mixed reality visualization device and overlaying the displayed three-dimensional image of the graft on or adjacent the anatomical features of the patient's bone to provide a visual guide to the surgeon while conducting surgery.

7. A method in accordance with claim 6 wherein the three-dimensional image of the graft is blended or merged with the initial three-dimensional image or the modified three-dimensional image.

8. A method in accordance with claim 1 wherein the pre-operative planning step further comprises generating a guidewire trajectory, and displaying the guidewire trajectory, via the mixed reality visualization device, overlayed on the displayed modified three-dimensional image to provide a visual guide to a surgeon while conducting surgery.

9. A system for visualization during orthopaedic surgery, the system comprising:
a processor operable to carry out image processing; and
a mixed reality visualization device which receives the modified three-dimensional image;
wherein the processor:
receives an initial three-dimensional image depicting anatomical features of a patient's bone, the initial three-dimensional image being retrieved by undertaking imaging or scanning of the patient's bone;
processes the initial three-dimensional image with one or more image processing steps to generate a modified three-dimensional image; and
communicates the modified three-dimensional image to the mixed reality visualization device; and
wherein the mixed reality visualization device displays the modified three-dimensional image and overlays the modified three-dimensional image on or adjacent the anatomical features of the patient's bone to provide a visual guide to a surgeon while conducting surgery.

10. A system in accordance with claim 9 wherein the mixed reality visualization device displays the initial three-dimensional image and overlays the displayed initial three-dimensional image on or adjacent the anatomical features of the patient's bone to provide a visual guide to a surgeon while conducting surgery.

11. A system in accordance with claim 9 wherein the processor analyses metal artefacts in the initial three-dimensional image and suppresses visualization of the metal artefacts and generates the modified three-dimensional image of the bone.

12. A system in accordance with claim 11 wherein analysing metal artefacts in the initial three-dimensional image further comprises the processor applying a mask to an initial two-dimensional image and reducing scatter of the initial two-dimensional image.

13. A system in accordance with claim 12 wherein reducing scatter of the initial two-dimensional image further comprises the processor applying a filter and/or modifying a filter strength of the filter applied to the initial two-dimensional image.

14. A system in accordance with claim 9 wherein the processor:
analyses a defect visible in the initial three-dimensional image;
generates geometric dimensions for a bone graft to fill said defects; and
generates a three-dimensional image of the graft; and
wherein the mixed reality visualization device displays the three-dimensional image of the graft and overlays the displayed three-dimensional image of the graft on or adjacent the anatomical features of the patient's bone to provide a visual guide to the surgeon while conducting surgery.

15. A system in accordance with claim 14 wherein the processor blends or merges the three-dimensional image of the graft with the initial three-dimensional image or the modified three-dimensional image.

16. A system in accordance with claim 9 wherein the processor generates a guidewire trajectory and the mixed reality visualization device displays the guidewire trajectory overlayed on the displayed modified three-dimensional image to provide a visual guide to a surgeon while conducting surgery.

17. A system in accordance with claim 9, wherein the processor is operable to receive user input via a user input interface to carry out the one or more image processing steps to generate the modified three-dimensional image.
